SEQUENCE LISTING (8 sequences)

SEQ ID NO: 1 - DNA, 20 bp, Artificial Sequence
  Description of Artificial Sequence Synthetic primer
ccccttcagccagcatataa

SEQ ID NO: 2 - DNA, 20 bp, Artificial Sequence
  Description of Artificial Sequence Synthetic primer
ttctctgggctgtctggact

SEQ ID NO: 3 - DNA, 20 bp, Artificial Sequence
  Description of Artificial Sequence Synthetic primer
tggagccatgattaggaagg

SEQ ID NO: 4 - DNA, 20 bp, Artificial Sequence
  Description of Artificial Sequence Synthetic primer
taaggaaggaccccatttcc

SEQ ID NO: 5 - DNA, 20 bp, Artificial Sequence
  Description of Artificial Sequence Synthetic primer
tgctgcttcatgttctgtga

SEQ ID NO: 6 - DNA, 20 bp, Artificial Sequence
  Description of Artificial Sequence Synthetic primer
tggtgcattagctttggttg

SEQ ID NO: 7 - DNA, 21 bp, Artificial Sequence
  Description of Artificial Sequence Synthetic primer
aaccacaggaacaaaacctca

SEQ ID NO: 8 - DNA, 20 bp, Artificial Sequence
  Description of Artificial Sequence Synthetic primer
cctcctgttggacatttgga

(19) United States
(12) Patent Application Publication
(10) Pub. No.: US 2021/0047699 A1
(43) Pub. Date: Feb. 18, 2021

(54) EARLY GENETIC SCREENING TO AID IN THE SELECTION OF DOGS FOR ASSISTANCE TRAINING PROGRAMS

(71) Applicants: The Trustees of Princeton University, Princeton, NJ (US); Oregon State University, Corvallis, OR (US)

(72) Inventors: Bridgett vonHoldt, Lawrenceville, NJ (US); Monique Udell, Albany, OR (US)

(73) Assignees: The Trustees of Princeton University, Princeton, NJ (US); Oregon State University, Corvallis, OR (US)

(21) Appl. No.: 16/990,468

(22) Filed: Aug. 11, 2020

Related U.S. Application Data
(60) Provisional application No. 62/887,892, filed on Aug. 16, 2019.

(51) Int. Cl. C12Q 1/6888; G16B 20/10; A01K 15/02; A61B 5/16; A61B 5/00

(52) U.S. Cl. CPC C12Q 1/6888; G16B 20/10; A01K 15/02; A61B 2503/42; A61B 5/7275; C12Q 2600/124; A61B 2503/40; A61B 5/165

(57) ABSTRACT

Disclosed herein is early genetic screening to aid in the selection of dogs for assistance training programs. Disclosed methods include a method for predicting the probability of canine success in a training program, involving genotyping a biological sample from a canine; determining at least one mobile element insertion copy number within the Williams-Beuren Syndrome (WBS) locus on canine chromosome 6; and predicting the probability of the canine's success in a training program based on the at least one mobile element insertion copy number. Another disclosed method is a method of producing dogs that are more likely to exhibit a sociable behavior, involving providing a male and female dog for breeding; determining at least one mobile element insertion copy number within the Williams-Beuren Syndrome (WBS) locus on canine chromosome 6 for each of the provided dogs; and mating the dogs of step (a) to produce offspring.

### CROSS-REFERENCE TO RELATED APPLICATIONS

100011 This application claims priority to U.S. Provisional Application Ser. No. 62/887,892, filed Aug. 16, 2019, which is herein incorporated by reference in its entireties for all purposes.

### REFERENCE TO A SEQUENCE LISTING

This application contains a Sequence Listing in computer readable form. The sequence listing consists of a file named “PRIN-70302_ST25.txt,” (2,130 bytes), created on Aug. 7, 2020. The computer readable form is incorporated herein by reference.

### TECHNICAL FIELD

Genetic screens for predicting the probability of a canine's success in an assistance training program are disclosed.

### BACKGROUND

Genome-wide approaches have increased in their application for exploring the molecular basis of animal behavior and personality. In contrast to many dog morphological traits, which have been successfully mapped to genetic variants, efforts to understand the causal genetic variants of dog temperament and personality are relatively new. Such insights can have positive implications for decisions regarding animal health and welfare. Although behavioral assays are currently used to identify extreme behavioral conditions in dogs, these methods are sensitive to the testing environment (e.g., stress behaviors are often documented in recently captured or relinquished shelter dogs) and may not be highly predictive of long-term positive behaviors. Additionally, as dog personality varies with age, behavioral assays likely face reduced accuracy for predicting adult behavior when conducted during sub-adult developmental stages. These limitations are compounded by the reliance of matching behavioral assays with the targeted dog behavioral traits, often with behavioral observations recorded by a non-specialist who has spent limited time with the study subject. Consequently, there is an immediate need for the development of an accurate, unbiased behavioral assessment technique.

The Canine Behavioral Assessment Research Questionnaire (C-BARQ) is a popular and validated questionnaire-based behavioral assessment tool used by pet owners, breeders, and trainers to evaluate their dogs. The C-BARQ describes dog behavior with respect to temperament, fear, trainability, and anxiety.

However, C-BARQ fails to provide accurate predictions of heritable behavior in adult dogs. This can be seen when considering training for assistance dogs. Assistance dogs are tremendously valuable with respect to their owner's physical and mental wellbeing. These include guide dogs (those that assist people with visual disabilities), hearing dogs (those that assist people with hearing impairments), and service dogs (those that assist people with motor disabilities). Assistance dog training programs explicitly survey behaviors that are imperative to their program's success, and include robustness to environmental stressors, social behaviors, trainability, attention, and problem-solving. However, these programs often document up to 60% failure rates, with behavioral problems reported as one of the primary causes for dismissal. For instance, 60% of dogs that are withdrawn from the Guide Dogs for the Blind program for behavioral reasons that include high activity level, incompatibility with cats or other dogs, and assertiveness toward leadership. Studies have identified that human-directed sociability behaviors, such as docility and fear towards strangers, have a profound effect in predicting success at assistance dog training programs. Additionally, dogs that fail assistance dog training programs often score significantly lower on C-BARQ behaviors that correspond with human-directed sociability. While the accuracy of behavioral assessment tools ranges from 64% and 87%, large variability exists in the positive predative value of assistance dog performance (8.4-93.3%), a measure that quantifies the likelihood that dogs classified as unsuccessful will be subsequently dismissed from training programs. An age-independent genetic assay that is informative for specific sociability-related behavioral traits could aid in efforts to identify candidates with social profiles that are well-suited for an assistance role. This information could influence training or placement decisions early in the dog's development, something that could provide welfare, economic benefits, and positive impact on public health.

### BRIEF SUMMARY

A first aspect of the present disclosure is drawn to a method for predicting the probability of canine's success in a training program. The method comprises three general steps: (i) genotyping a biological sample from a canine; (ii) determining at least one mobile element insertion copy number within the Williams-Beuren Syndrome (WBS) locus on canine chromosome 6; and (iii) predicting the probability of the canine's success in a training program based on the at least one mobile element insertion copy number. The biological sample be, e.g., blood, saliva, cerebrospinal fluid, skin, or urine.

Optionally, genotyping the biological sample includes PCR amplification and agarose gel electrophoresis. Optionally, genotyping the biological sample utilizes at least one primer selected from the group consisting of: (i) CCCCTTCAGCCAGCATATAA [SEQ ID NO:1]; (ii) TTCTCTGGGCTGTCTGGACT [SEQ ID NO:2]; (iii) TGGAGCCATGATTAGGAAGG [SEQ ID NO:3]; (iv) TAAGGAAGGACCCCATTTCC [SEQ ID NO:4]; (v) TGCTGCTTCATGTTCTGTGA [SEQ ID NO:5]; (vi) TGGTGCATTAGCTTTGGTTG [SEQ ID NO:6]; (vii) AACCACAGGAACAAAACCTCA [SEQ ID NO:7]; and (viii) CCTCCTGTTGGACATTTGGA [SEQ ID NO:8].

Optionally, the mobile element insertions interrupt a gene in the WBS locus. Optionally, the mobile element insertions are retrotransposon mobile element insertions. Optionally, the method according to claim 6, wherein the retrotransposons are short interspersed nuclear elements (SINEs) or a long interspersed nuclear elements (LINEs). Optionally, the at least one mobile element insertion occurs within at least one gene selected from the group consisting of GTF2I, POM121, and WBSCR17.

Optionally, predicting the probability of the canine's success in a training program includes rating the canine on attachment/attention-seeking behaviors and separation-related problems. Optionally, the rating for attachment/attention-seeking behaviors is based on Cfa6.6 and Cfa6.83, and the rating for separation-related problems is based on Cfa6.6 and Cfa6.7. Optionally, which loci are used to determine each rating is based on age of the canine. Optionally, the probability of the canine's success in a training program is further based on the heterozygosity deficiency at locus Cfa6.6.

Optionally, at least one mobile element insertion is found at Cfa6.6, Cfa6.7, Cfa6.66, or Cfa6.83. In some instances, at least one mobile element insertion is found at Cfa6.6 and Cfa6.7. Optionally, the probability of the canine's success in a training program is not based on a mobile element insertion copy number of loci Cfa6.66.

In some instances, the probability of the canine's success in a training program is further based on an age of the canine.

A second aspect of the present disclosure is drawn to a method of producing dogs that are more likely to exhibit a sociable behavior. determining at least one mobile element insertion copy number within the Williams-Beuren Syndrome (WBS) locus on canine chromosome 6 for biological samples from a male and female dog intended for breeding; and allowing the male and female dog to produce offspring based on the determination of the at least one mobile element insertion copy number. Optionally, the dogs have a heterozygosity deficiency at locus Cfa6.6, and/or at least one mobile element insertion occurs within at least one gene selected from the group consisting of GTF2I, POM121, and WBSCR17.

### BRIEF DESCRIPTION OF FIGS.

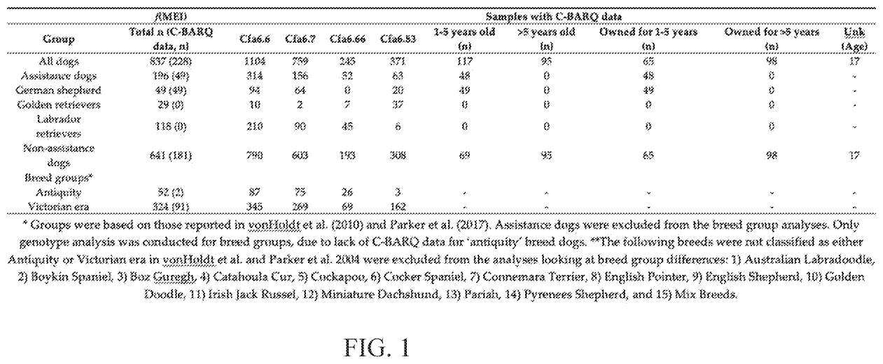

FIG. 1 is a table describing the number of dogs genotyped with associated C-BARQ behavioral data (Abbreviations: f, frequency; MEI, mobile element insertion; n, sample size; ND, no data; Unk, unknown). The Breed groups were based on those reported in vonHoldt et al. (2010) and Parker et al. (2017). Assistance dogs were excluded from the breed group analyses. Only genotype analysis was conducted for breed groups, due to lack of C-BARQ data for ‘antiquity’ breed dogs. The following breeds were not classified as either Antiquity or Victorian era in vonHoldt et al. and Parker et al. 2004 were excluded from the analyses looking at breed group differences: 1) Australian Labradoodle, 2) Boykin Spaniel, 3) Boz Guregh, 4) Catahoula Cur, 5) Cockapoo, 6) Cocker Spaniel, 7) Connemara Terrier, 8) English Pointer, 9) English Shepherd, 10) Golden Doodle, 11) Irish Jack Russel, 12) Miniature Dachshund, 13) Pariah, 14) Pyrenees Shepherd, and 15) Mix Breeds.

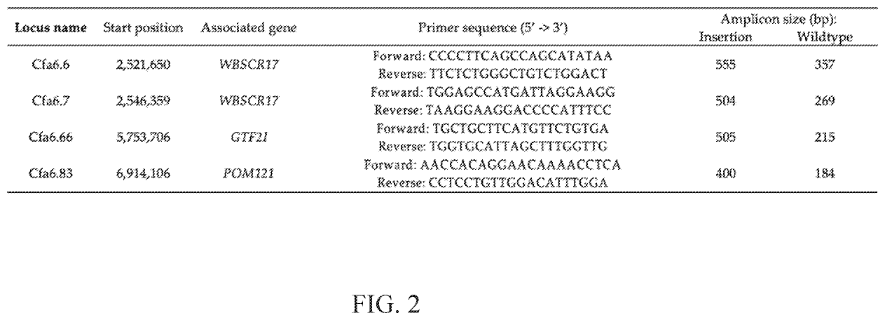

FIG. 2 is a table describing primer sequence and amplicon information for four retro-transposon mobile element insertions located on canine chromosome 6 in Canfam3.1. (Abbreviations: bp, base pair).

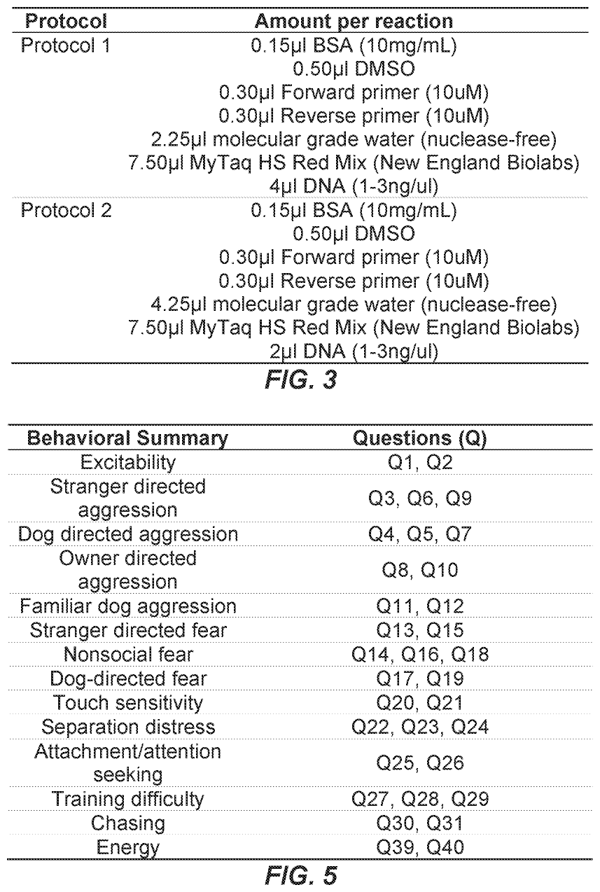

FIG. 3 is a table describing thermocycling reagents and concentrations for locus amplification using either Protocol 1 with cycling conditions: 95° C. for 10 min; 30 cycles of 95° C. for 30 s, 60° C. for 30 s, 72° C. for 45 s; 72° C. for 10 min; 4° C. hold or Protocol 2. Cycling conditions with cycling conditions: 95° C. for 5 min; 46 cycles of 94° C. for 1 min, 60° C. for 1 min, 72° C. for 1.5 min; 72° C. for 5 min; 4° C. hold. The desired PCR products are between 200 bp-500 bp. Protocol 1 amplifies loci with higher specificity, while Protocol 2 is used when DNA volume is limited.

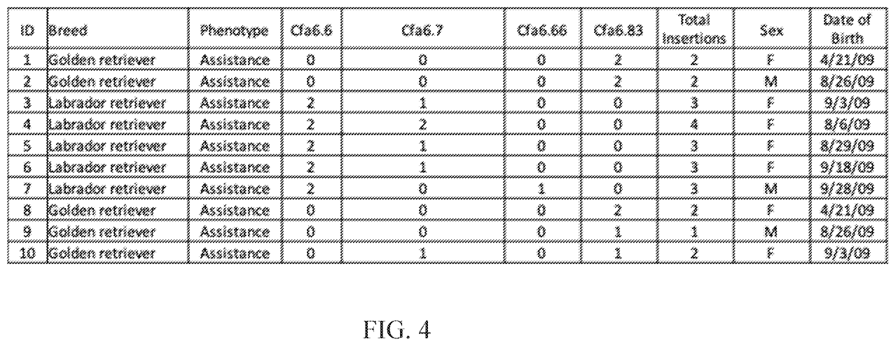

FIG. 4 is a table detailing the first 10 rows of data from the determination of mobile element insertion copy number within the WBS locus on canine chromosome 6 for different canines in the screening.

FIG. 5 is a table describing the calculation of C-BARQ behavior axes as per averaging scores for identified questions.

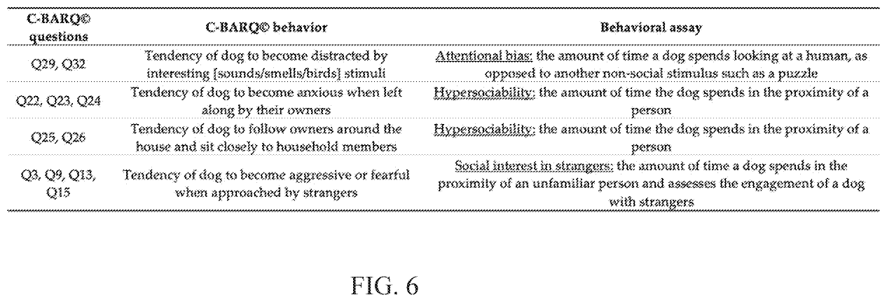

FIG. 6 is a table describing questions on the C-BARQ that were tested for overlap in behaviors found to be associated with canine hyper-sociability (Abbreviations: Q, question).

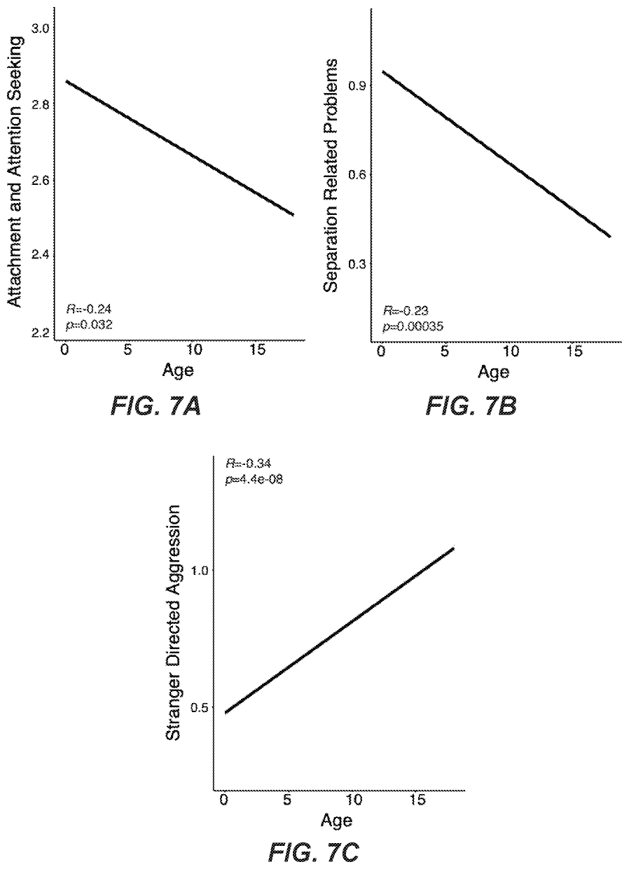

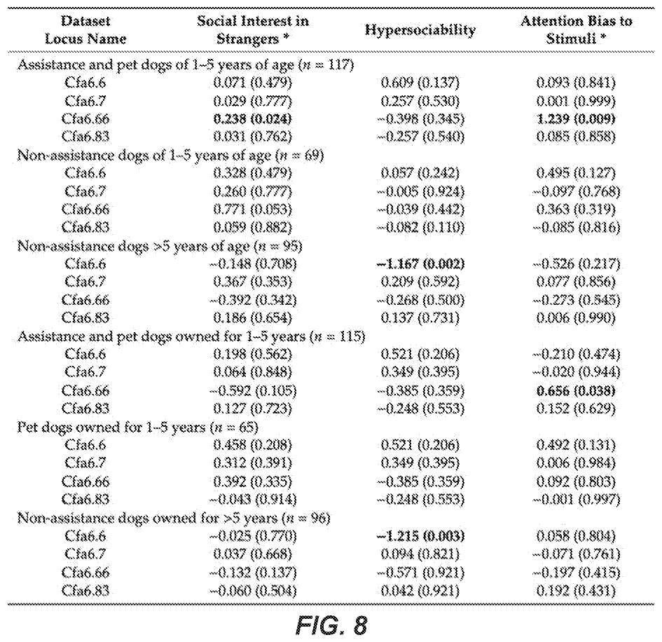

FIG. 7A is a graph showing the correlation between age and attachment and attention-seeking behavior.

FIG. 7B is a graph showing the correlation between age and separation-related problems.

FIG. 7C is a graph showing the correlation between age and stranger-directed aggression.

FIG. 8 is a table of per locus beta values as a function of MEI copy number and C-BARQ score averages that quantify behaviors associated with three aspects of hypersociability. Significance values are in parentheses (bolded values indicate p<0.05).

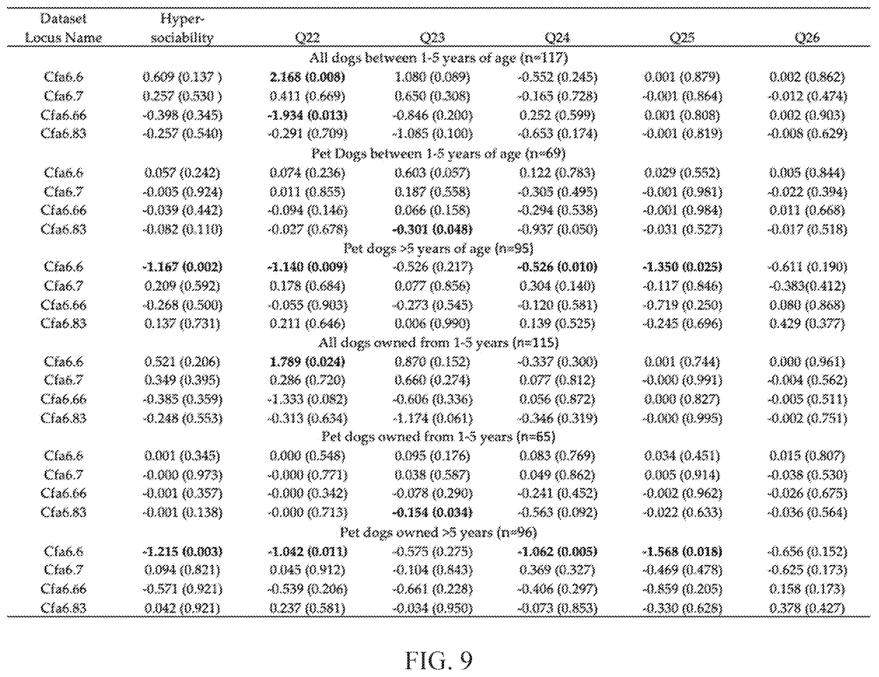

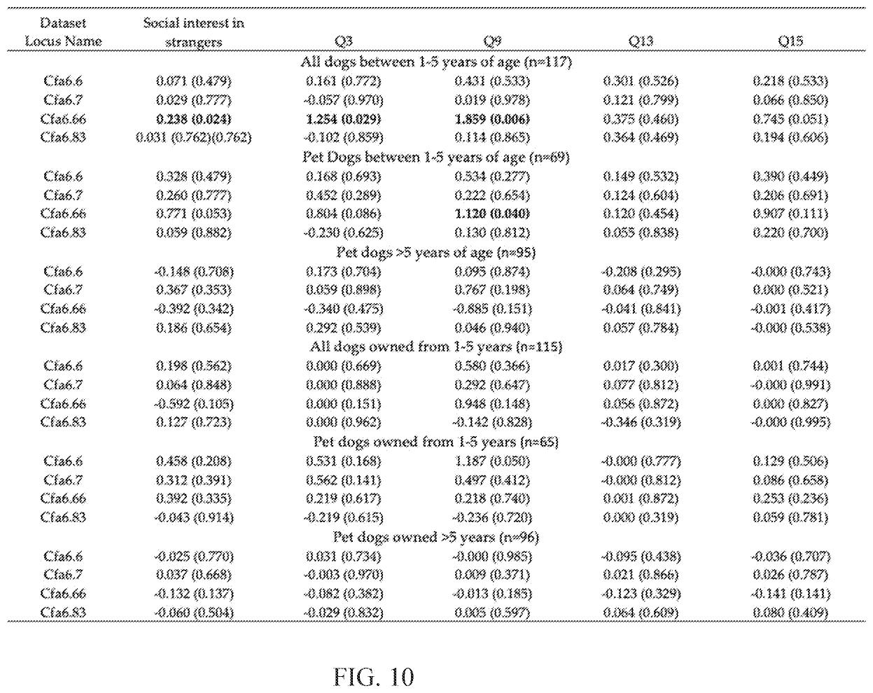

FIG. 9 is a table of beta values for locus-specific MEI copy number and C-BARQ score averages informative for hyper-sociability and for relevant question tagging this behavioral summary. P-values are in parentheses; bolded values indicate p<0.05. Beta values were obtained using linear ridge regression with age and sex as covariates.

FIG. 10 is a table showing beta values for locus-specific MEI copy number and C-BARQ score averages informative for social interest in strangers and for relevant question tagging this behavioral summary. P-values are in parentheses; bolded values indicate p<0.05. Beta values were obtained using linear ridge

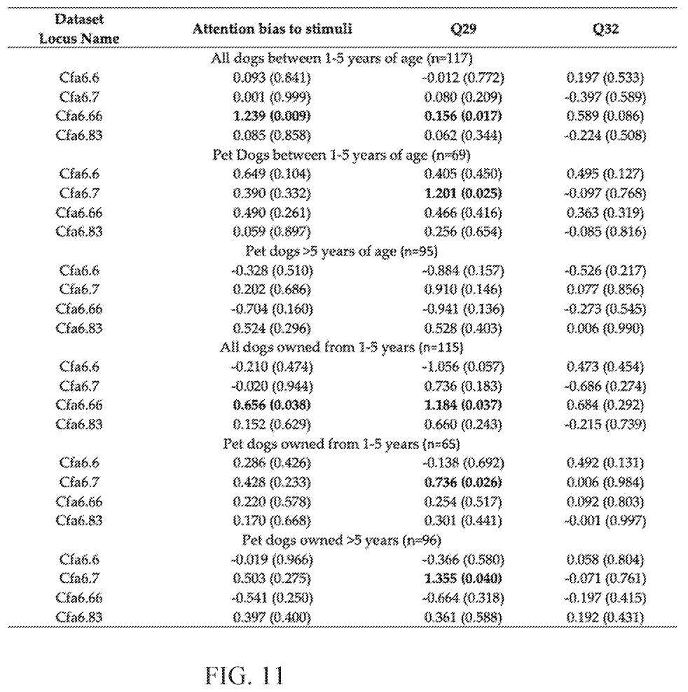

FIG. 11 is a table showing beta values for locus-specific MEI copy number and C-BARQ score averages informative for attention bias to stimuli and for relevant question tagging this behavioral summary. P-values are in parentheses; bolded values indicate p<0.05. Beta values were obtained using linear ridge regression, using age and sex as covariates.

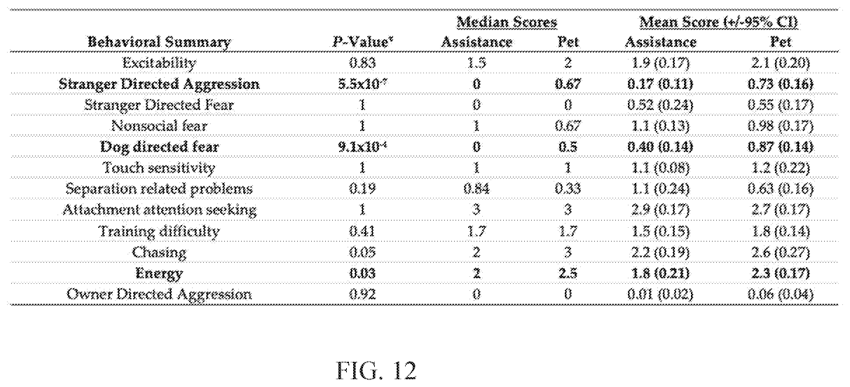

FIG. 12 is a table showing differences in scores between assistance dogs and pet dogs on the C-BARQ with respect to the defined behavioral axes. Bolded values indicate p<0.05. (Abbreviations: CI, confidence interval).

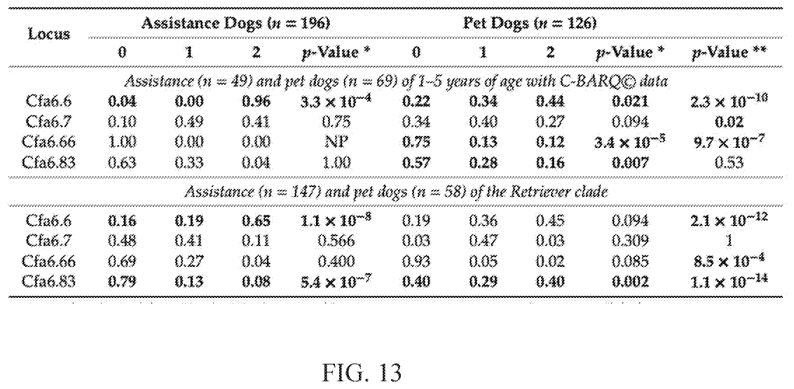

FIG. 13 is a table summarizing per-locus significance testing from Hardy—Weinberg equilibrium exact tests for assistance and pet dogs. Genotypes are represented by the number of MEIs (homozygous with no insertions, 0; heterozygous for insertion, 1; homozygous for insertion, 2). Bolded values indicate p<0.05. (Abbreviations: NP, test not performed).

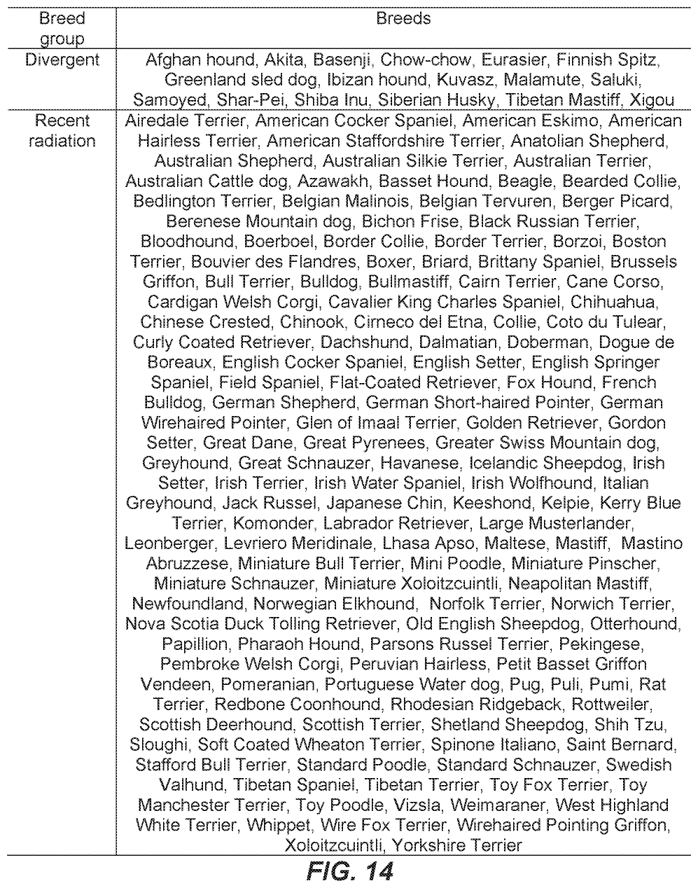

FIG. 14 is a table summarizing the classification of breed groups into “Divergent” or “Recent Radiation” groups.

### DETAILED DESCRIPTION

Disclosed are methods that generally involve the combinatorial addition of a genetic assay that is easy to conduct alongside the C-BARQ. These methods improve the prediction of heritable behavior in adult dogs. This involves the integration of an assessment tool, such as C-BARQ, with genotyping for specific targeted dog populations. The combination of a questionnaire and genetic assay reduces the time and effort required of other behavioral assessments, an aspect that may be especially beneficial when evaluating assistance dog candidates for entry into their respective time- and cost-intensive training programs.

Recently, four canine retrotransposon mobile element insertions (MEIs) were discovered in domestic dogs and gray wolves that were significantly associated with a heightened propensity to initiate prolonged social contact, commonly referred to as hypersociability. These MEIs are located on canine chromosome 6 (CFA6) and associated with the genes WBSCR17 (Cfa6.6 and Cfa6.7), GTF2I (Cfa6.66), and POM121 (Cfa6.83), which are known to be involved in Williams-Beuren syndrome in humans. MEIs within the genes WBSCR17 and POM121 were associated with increased hypersociability, while MEIs within the gene GTF2I were associated with decreased hypersociability. Additionally, higher copy number in WBSCR17 and POM121 were associated with increased breed groups by their stereotyped attention-seeking behaviors. These MEIs were also found to segregate within breeds as well as wolves, and significantly associated with changes in social behavior after accounting for species membership. These MEIs are retrotransposon elements, and hence are highly methylated. Through a survey of methylation and transcriptional data, these MEIs were found to affect gene expression likely via cis-regulatory pathways. MEIs are highly methylated and may regulate adjacent genes. These elements are easy to genotype through a targeted PCR protocol, and thus identifying MEI copy number at these four loci is straightforward.

The method for predicting the probability of canine success in a training program generally involves first genotyping a biological sample from a canine. The biological sample may comprise any appropriate DNA source, but preferably the biological sample is a blood, saliva, cerebrospinal fluid, skin, or urine sample. The biological samples are gathered in an appropriate manner as known by those of skill in the art. One preferred approach for acquiring the biological samples is via a buccal (cheek) swab.

Genotyping can be done in any acceptable manner as known by those of skill in the art. In a preferred embodiment, genotyping the biological sample includes PCR amplification and agarose gel electrophoresis. In some embodiments, genotyping the biological sample utilizes at least one primer selected from the group consisting of: (i) CCCCTTCAGCCAGCATATAA [SEQ ID NO:1]; TTCTCTGGGCTGTCTGGACT [SEQ ID NO:2]; (iii) TGGAGCCATGATTAGGAAGG [SEQ ID NO:3]; (iv) TAAGGAAGGACCCCATTTCC [SEQ ID NO:4]; (v) TGCTGCTTCATGTTCTGTGA [SEQ ID NO:5]; (vi) TGGTGCATTAGCTTTGGTTG [SEQ ID NO:6]; (vii) AACCACAGGAACAAAACCTCA [SEQ ID NO:7]; and (viii) CCTCCTGTTGGACATTTGGA [SEQ ID NO:8].

Once the sample has been genotyped, the method involves determining at least one mobile element insertion copy number within the Williams-Beuren Syndrome (WBS) locus on canine chromosome 6. In some embodiments, the mobile element insertions interrupt a gene in the WBS locus. In some embodiments, these mobile element insertions may be retrotransposon mobile element insertions. In some instances, the retrotransposons are short interspersed nuclear elements (SINEs). In some embodiments, the retrotransposons are long interspersed nuclear elements (LINEs). In some embodiments, the mobile element insertions may include one or more SINEs and one or more LINEs.

In preferred embodiments, at least one mobile element insertion is found at Cfa6.6, Cfa6.7, Cfa6.66, or Cfa6.83. In some embodiments, at least one mobile element insertion is found at Cfa6.6 and Cfa6.7.

In preferred embodiments, at least one mobile element insertion occurs within at one of the following genes: GTF2I, POM121, or WBSCR17. In some embodiments, the sample is free of MEIs within GTF2I. In some embodiments, the sample is free of MEIs within POM121. In some embodiments, the sample is free of MEIs within WBSCR17. In some embodiments, the mobile element insertions occur only within two of the three genes. In some embodiments, the MEIs occur within all three of the genes.

### EXAMPLE 1

Genotyping and Counting Four Mobile Element Insertions Associated with Human-Directed Hypersociability.

As part of a screen, DNA from 837 adult domestic dogs >1 year of age was isolated, 159 of which were whole blood samples and 678 from buccal cells or saliva using Qiagen's DNeasy Blood and Tissue Kit (Qiagen, Germantown, Md., USA). A summary can be seen in FIG. 1. The samples were derived across 74 breeds (n: purebred=656, mixed-breed=104, unknown=78), from 196 assistance dogs (sample size per breed: German Shepherds=56, Golden Retriever=29, Labrador Retriever=118) and 642 pet dogs. For all DNA isolated from buccal cells or saliva, a second purification step was completed using a 1:2 ratio of DNA to AMPure XP magnetic purification beads (Beckman Coulter Life Sciences, Indianapolis, Ind., USA). Previously published amplification methods were followed to genotype and survey the insertional dynamics of four MEIs implicated in canine hypersocial behavior. Amplicons between 215 bp and 555 bp in length were obtained. The total PCR product was visualized on a 1.8% agarose gel for scoring genotypes as the number of insertions per locus per individual (0, 1, or 2). FIG. 2 describes an example of primer sequence and amplicon information for four retro-transposon mobile element insertions located on canine chromosome 6 in Canfam3.1. FIG. 3 provides an example of thermocycling reagents and concentrations for locus amplification for 2 protocols. Protocol 1 amplifies loci with higher specificity, while Protocol 2 is used when DNA volume is limited. FIG. 4 shows the first 10 rows of data from the determination of mobile element insertion copy number within the WBS locus on canine chromosome 6 for different canines in the screening.

The method continues by predicting the probability of the canine's success in a training program based on the at least one mobile element insertion copy number. Generally, a correlation is determined between the MEIs (including, e.g., the number and/or the location of the MEIs) and a behavioral assessment (such as, e.g., C-BARQ) that identifies behaviors of interest that lead to success in the training program. Once a correlation is identified, the probability of success can be predicted based on the, e.g., the MEIs in a given sample.

In some embodiments, these predictions involve rating the canine on attachment/attention-seeking behaviors and separation-related problems. In some embodiments, the rating for attachment/attention-seeking behaviors is based on Cfa6.6 and Cfa6.83, and the rating for separation-related problems is based on Cfa6.6 and Cfa6.7.

In some embodiments, which loci are used to determine each rating is based on age of the canine.

In some embodiments, the probability of the canine's success in a training program is not based on a mobile element insertion copy number of loci Cfa6.66.

In some embodiments, the probability of the canine's success in a training program is further based on the heterozygosity deficiency at locus Cfa6.6.

In some embodiments, the probability of the canine's success in a training program is further based on an age of the canine.

### EXAMPLE 2

Canine Questionnaire Data to Identify Behavioral Types

Of 837 dogs with genetic samples, 228 also had paired detailed demographic (age, sex, breed, DOB, and # years owned) and behavioral data derived from 42 questions of the C-BARQ (short version). As understood by those of skill in the art, the C-BARQ quantifies the behavioral tendencies of individual dogs (as assessed by an owner, handler, or evaluator) across 14 behavioral categories by averaging scores across related questions (See FIG. 5). Questions from the attachment, attention-seeking, and separation distress sections of the evaluation most closely parallel behavioral traits used to identify dogs displaying hypersociability (elevated proximity-seeking) in prior research by vonHoldt et al. (See FIG. 6). Additionally, stranger-directed aggression and stranger-directed fear quantify opposing behaviors towards unfamiliar people, and hence would be negatively correlated with prosocial interest in strangers (See FIG. 6). Questions from the trainability and chasing sections (questions Q29 and Q30, respectively) closely match the attention bias to stimuli behavioral summary; however, the C-BARQ questionnaire does not specify the nature of the stimulus in terms of being social or nonsocial (See FIG. 6).

Previous studies have shown that dog behavior varies with age. Spearman's rank correlational tests were conducted to elucidate associations between dog age and sociability measures on the C-BARQ. Four datasets of pet dogs were created based on demographics from the C-BARQ to ensure that differences in behavior that may be due to the age of the dog or how many years the dog was owned at the time of reporting were accounted for. Each of these datasets has paired MEI genotype and behavioral C-BARQ data. The datasets comprising of pet dogs were as follows: (1) 69 young adult dogs of 1-5 years of age; (2) 95 adult dogs over >5 years of age; (3) 65 dogs that have been owned for between 1 and5 years by the reporting owner; and (4) 98 dogs that have been owned for >5 years by the reporting owner (See FIG. 1). Additionally, this dataset included 49 German Shephard assistance dogs, all of 1-5 years of age, with paired genotype and C-BARQ data. Some samples lacked data regarding the number of years a dog had been owned and date of birth. These samples were excluded from all subsequent analyses, whereby age is a relevant stratification factor.

Using the Spearman correlational tests to evaluate age based trends in the C-BARQ behavioral axes, it was found that on average, an increase in age (age range: 1-17 years; median age: 10 years) corresponded to a decrease in attachment and attention-seeking behavior (FIG. 7A) (R=−0.24, p=0.03), a decrease in separation-related problems (FIG. 7B) (R=−0.23, p≥0.001), and an increase in stranger-directed aggression (FIG. 7C) (R=0.34, p≥0.001). See FIGS. 7A-7C.

Thus, in some embodiments, taking the age of the canine into account may be useful.

To elucidate the potential order of importance of these loci in affecting sociability-related phenotypes, conditional random forests ensemble algorithms were employed using 20,000 bootstrap samples from the R package “party” (Hothorn et e.g., 2006) and function “party” for all dogs of 1-5 years of age. C-BARQ behavioral axes related to attachment-seeking and separation distress behaviors were used as response variables. Conditional random forests were used to alleviate confounding importance measures due to interdependency of the loci. Variable importance measures (VARIMP) were computed using the function “varimp”, setting the conditional parameter to “true”. Variables with a positive VARIMP increase predictive power, while variables with a negative VARIMP decrease predictive power. A large positive VARIMP indicates a potentially predictive variable. Two types of models were analyzed: (1) the inclusion of all loci, with each as a predictor; and (2) the exclusion of locus Cfa6.66 due to its low interindividual variation.

The highest positive variable importance for attachment/attention-seeking (VARIMP=0.003) and found the highest positive variable importance for attachment/attention-seeking (VARIMP=0.003) separation-related problems (VARIMP=0.04) were found at locus Cfa6.6. Locus Cfa6.7 had negative variable importance for attachment/attention-seeking (VIMP=−0.002) and positive variable importance for separation-related problems (VARIMP=0.003), while locus Cfa6.83 had moderate positive variable importance for attachment/attention-seeking (VARIMP=0.004) and separation-related problems positive variable importance for attachment/attention-seeking (VARIMP=0.004) and separation-(VARIMP=0.001). When Cfa6.66 is included in the model, Cfa6.6 still has the highest variable importance (VARIMP=0.012) for dogs of 1-5 years of age, but none of the loci have predictive power for attachment/attention-seeking. Thus, in some embodiments, depending on the age of the dog, certain loci may or may not be useful in aiding in predictions. For example, in some embodiments, Cfa6.66 is not considered when making a prediction, while in other embodiments, Cfa6.66 is not considered when making a prediction only if the canine is between 1-5 years of age.

Previously, hypersocial canine behavior was quantified using solvable tasks and sociability measures, including dogs' propensity to spend time looking at a human relative to a nonsocial stimulus (referred to as attentional bias) and their propensity to spend time in proximity to familiar or unfamiliar humans (hypersociability and social interest in strangers, respectively). To determine which personality axes of the C-BARQ are predicted by the MEI genetic test, four datasets of pet dogs were constructed to control for age and the number of years the dog had been owned. Two additional datasets were created which include both pet and assistance dogs for dogs of age 1-5 years (n=117) and those owned from 1-5 years (n=115), since all assistance dogs containing C-BARQ scores belonged to these groups. The associations between MEI copy number and the C-BARQ questions hypothesized to be informative for social behavior were modeled. The associations between MEI copy number and C-BARQ scores using linear ridge regression were measured. Ridge regression considers the correlation between predictor variables and corrects for multicollinearity. The datasets using the R package “ridge” (Cule et al., 2013) were analyzed and implemented identical parameter settings for each dataset to control for age and number of years owned. Age and sex were included as covariates in the models. Beta values and p-values were estimated for each relevant question and for the average scores across all questions tagging the same general behaviors as per vonHoldt et al., 2017, which include hypersociability (average of Q22, Q23, Q24, Q25, and Q26), social interest in strangers (average of Q3, Q9, Q13, and Q15), and attention bias to social stimuli (average across Q29 and Q32). Higher averages on Q3, Q9, Q13, Q15 and Q29, Q32 correspond to lower social interest in strangers and attention bias to stimuli, respectively.

Differences in C-BARQ behavioral scores and MEI copy number were assessed comparing assistance and pet dogs. Differences in C-BARQ behavior and MEIs were assessed using Mann-Whitney U tests with a Bonferroni correction implemented with the R functions wilcox.test and “p.adjust”. To assess whether each group experienced heterozygosity deficiency at a locus, within-group differences in heterozygosity (observed, HO) were estimated, and allele frequencies were estimated with the Hardy-Weinberg equilibrium (HWE) exact test function “Hwe.exact” in the R “genetics” package. Between-group differences were analyzed using the Bonferroni-corrected Fisher's exact test (“fisher.exact”). Paired C-BARQ scores and genotype data were present for assistance dogs belonging to a single breed, the German Shepherd. Consequently, the analysis was repeated for genotypic differences of pure-bred dogs belonging to a single breed clade called Retrievers to ensure, at a certain extent, that genotypic differences detected in the previous analysis are not breed-specific and instead truly represent differences between assistance and pet dogs, regardless of breed.

It was determined that higher Mobile Element Copy Numbers at Cfa6.6 Co-Occur with Increased Hypersociability in Younger Dogs. Higher MEI copy number at Cfa6.6 is associated with decreased hypersociability for dogs of age >5 years (b=−1.167; p=0.002) and those owned for >5 years (b=−1.215; p=0.003) (See FIG. 8). Contrary to this pattern, pets of age 1-5 years (b=0.057; p=0.242) and those owned from 1-5 years (b=0.521; p=0.206), have higher yet nonsignificant associations for MEI copy number at Cfa6.6 and increased hypersociability (FIG. 8). When considering all dogs (assistance and pet) from ages 1-5, higher MEI copy number at Cfa6.6 is significantly associated with increased scores on hypersociability-related Q22 of the C-BARQ (b=2.168; p=0.008) (See FIG. 9). Higher MEI copy number at Cfa6.66 is significantly associated with higher scores on Q9, which is associated with increased aggression towards strangers (b=1.120, p=0.040) (See FIG. 10). This association is also apparent for Q9 among assistance and pet dogs of 1-5 years of age (b=1.859, p=0.006), in addition to Q3 (b=1.254, p=0.029) (See FIG. 9) and the overall behavioral summary of lower social interest in strangers (b=0.238, p=0.024) (See FIG. 8). Higher MEI copy number at Cfa6.66 is associated with lower attentional bias on Q29 for pet dogs of age 1-5 years (b=1.201; p=0.025) and those owned from 1-5 years (b=0.736, p=0.029) (See FIG. 11). This association with Q29 was also apparent for assistance and pet dogs of age 1-5 years (b=0.156, p=0.017) and those owned from 1-5 years (b=1.184; p=0.037) (See FIG. 11), in addition to the overall behavioral summary of lower attention bias to stimuli (age 1-5 years: b=1.239, p=0.009; owned from 1-5 years: b=0.656, p=0.038) (See FIG. 8).

It was found that Assistance Dogs Consistently Have More Mobile Element Insertions and Significant Heterozygosity Deficiency at Cfa6.6 Compared to Non-Assistance Dogs. German Shepherd assistance dogs of 1-5 years of age (n=49) had significantly lower median scores on C-BARQ questions negatively associated with social interest in strangers than pet dogs of the same age group (n=69): stranger-directed aggression (median score: assistance=0.0, pet=0.7, p=5.5×110⁻⁷; range: 0-4), dog-directed fear (median score: assistance=0.0, pet=0.5, p=9.1×10⁻⁴; range: 0-4), and energy, which measures a dogs' propensity to act in a playful, boisterous, and active manner (median score: assistance=2.0, pet=2.5, p=0.03; range: 0-4). Assistance dogs had higher, yet non-significant, average C-BARQ scores related to separation distress relative to pet dogs (average score: assistance=0.8, pet=0.3, p=0.19; range: 0-4) (See FIG. 12).

Assistance dogs had lower observed heterozygosity (H_{O}=0.08, p=3.2×10⁻⁴) relative to pet dogs at loci Cfa6.6 (H_{O}=0.48, p=3.4×10⁻⁴), where the proportion of observed frequency of the insertion allele was 96% in assistance dogs, compared to 61% in pet dogs (Bonferroni-adjusted p=2.3×10⁻¹⁰). Additionally, assistance dogs had significantly more MEIs at Cfa6.7 (Fisher's exact test; Bonferroni-adjusted p=0.02) (approximately 63% in assistance dogs as compared to 40% in pet dogs). Further, German Shepherd assistance dogs had no alleles containing insertions at Cfa6.66, compared to the 20% MEI allele frequency in pet dogs (Fisher's exact test; Bonferroni-adjusted p=9.7×10⁻⁷). Retriever-clade assistance dogs had a lower heterozygosity (H_{O}=0.378, p=1.1×10⁻⁸) relative to pet dogs at locus Cfa6.6 (H_{O}=0.471, p=0.094). In comparison to pet Retriever-clade dogs (Labrador Retriever, n=9; Golden Retriever, n=48), assistance Retriever-clade individuals had a proportionally higher frequency of MEIs at Cfa6.6 (assistance=75%, pet=37%, Bonferroni-corrected p=8.3×10⁻¹²) and Cfa6.66 (assistance=18%, pet=4%, Bonferroni-corrected p=8.5×10⁻⁴), and lower frequency of MEIs at Cfa6.83 (assistance=15%, pet=54%, Bonferroni-corrected p=1.1×10⁻¹⁴). For both sets of assistance dogs, the population were not in HWE and presented an excess of genotypes homozygous for the MEI at Cfa6.6 (See FIG. 13).

MEIs may display allele frequency differences for breeds with respect to their genetic relationships with other breeds and their own unique breed history. Dogs may be grouped into breed groups based on previous studies that identified the genetic relationships between breeds using phylogenetic trees. Several studies have reported breed groups that are highly genetically divergent and distinct. The primary signals of divergence are from Asian Spitz-type breeds, Arctic Spits-type breeds, Sighthounds, and African breeds, which branched separately from European-derived breeds. Many of these aforementioned breed groups have histories that consider them ancient in origins compared to those known to have a more recent establishment with subsequent rapid radiation, although both breed groups have experienced augmentation throughout their existence. Following these previously studies, these genetically distinct breeds can be grouped in a category called divergent and the remainder of breeds into the recent-radiation category.

The MEI insertion frequency in dogs belonging to either divergent or recent-radiation breed groups was assessed. It is suspected that selection for human-directed hypersociability pre-Victorian-era breed radiation likely resulted in differences in sociability and MEI frequencies before and after the Victorian breed radiation. As the recent radiation rapidly developed numerous new breeds with targeted functions, it is suspected that many breeds were not selected explicitly for hypersociability and the associated MEIs likely drifted or were purged. Dog breeds were categorized regarding their documented origins (n, divergent=52, recent radiation=324) following previously published classifications (FIG. 14). Within-group genotype and allele frequency differences were assessed using the Hardy-Weinberg equilibrium (HWE) exact test using the function “Hwe.exact” on the R package “genetics”. Between-group allele frequency differences were analyzed using the Bonferroni-corrected Fisher's exact test (“fisher.exact”). Assistance dogs were excluded from these analyses. As divergent breeds represented a smaller fraction of the overall dataset (n=52), 100 individuals from the recent-radiation group were randomly sampled to ensure sample size differences did not introduce any biases. Only pure-bred dogs were used for this analysis.

Divergent dog breeds, as compared to the recent radiation dog breeds, had higher frequency of inserted alleles at Cfa6.6 (˜85% vs. ˜50%) (Bonferroni-adjusted p=3.6×10⁻⁷), Cfa6.7 (˜70% vs. ˜38%) (Bonferroni-adjusted p=6.4×10⁻⁷), and Cfa6.66 (Bonferroni-adjusted p=8.0×10⁻⁷) (˜25% vs. ˜10%); however, the opposite pattern was noted for locus Cfa6.83 (Bonferroni-adjusted p=8.8×10⁻⁸) (˜5% vs. ˜28%). Additionally, a significant deficiency of heterozygosity was found for divergent dog breeds for loci Cfa6.6 (H_{O}=0.276) and Cfa6.83 (H_{O}=0.057), due to higher frequency of homozygous genotypes for the insertion at Cfa6.6 (p=1.6×10⁻³) and homozygous genotypes for the no insertions at Cfa6.83 (p=0.029).

As one of skill in the art will recognize based on the above, there are numerous ways to predict the probability of success for a canine in a training program. In some embodiments, a computer model will be built based on the predetermined correlations between target behaviors and MEIs. A computer can then receive the necessary MEI information for a particular canine from a user (e.g., typing the information in on a keyboard, uploading a file containing the information to the computer, or causing another analytical instrument to report the data directly to the computer, etc.). The computer can then run the necessary MEI information through the model, and output a predicted probability of success. In some embodiments, this includes a descriptive prediction (e.g., “Very Likely”, “Likely”, “Unlikely”, “Very Unlikely”, etc.), while in others it includes a numerical prediection (e.g., “82% chance of success”).

The above-disclosed highly predictive genetic markers for social behaviors relevant to assistance dog training and placement may provide important benefits, including increased success rates, lower costs, and early behavioral interventions, allowing for better welfare and greater assistant dog availability. Locus Cfa6.6, a locus associated with WBSCR17, has the highest predictive power for C-BARQ behaviors related to the hypersocial phenotype, which include higher levels of attention-seeking and separation-related distress among adult dogs of age 1-5 years. Increased frequencies of insertions at Cfa6.6 was associated with higher scores on the hypersociability-related C-BARQ question Q22, which is consistent with previous work. This question pertains to separation distress and assesses a dog's restlessness, agitation, or pacing when about to be left alone by its owner.

One of the behavioral traits of successful assistance dogs include increased sociability towards people with disabilities. Assistance dogs, of both German Shepherd and Retriever clades, showed reduced heterozygosity at Cfa6.6 relative to pet dogs due to the significant increase in the frequency of the insertion allele. Further, dogs homozygous for the insertion at Cfa6.6 also show increased human-directed hypersociability as quantified by separation distress and decreased human-opposed behavior such as stranger-directed aggression. These analyses show higher frequency of MEIs in assistance dogs. Genetic screening of dogs for insertions at, e.g., WBSCR17 may assist in identifying social predispositions of dogs early in development, which could increase successful training and placement of assistance dogs.

A higher frequency of MEIs was found at WBSCR17 within divergent dog breeds. This finding is also consistent with the original discovery that WBSCR17 contains genetic variation that differentiates domestic dogs from gray wolves, suggesting that behavioral traits were an important selection factor for dog domestication.

While not a complete replacement for behavioral evaluations or interventions, genetic screening may provide an additional assessment tool to aid in the optimal placement, care, and training of animals based on underlying behavioral predispositions that may not otherwise be detectable early in life. Such a tool may be especially beneficial when evaluating working dog candidates, such as assistance dogs, for selective enrollment into resource-intensive training programs. From these analyses, it is seen that higher MEI copy number at WBSCR17 is associated with C-BARQ hypersociability behaviors and that these traits may be prominent in the tested populations of active and successful assistance dogs. As a result, genetic screening for these hypersociability insertions may be a valuable tool in the early screening or breeding of assistance dogs.

A second aspect of the disclosed method is drawn to a method of producing dogs that are more likely to exhibit a sociable behavior. This method builds on the above-described methodology, but does not necessarily require generating any correlations between the genotype and the assessment tool.

Assuming one had genotyped a male and a female dog, after the mobile element insertion copy numbers within the Williams-Beuren Syndrome (WBS) locus on canine chromosome 6 have been determined, the male and female dogs can be mated to produce offspring. In some embodiments, the mating is allowed to occur after predicting the probability of canine success in a training program for both dogs, as described above. In some embodiments, the probability of success for both dogs should be above a predetermined threshold (such as ≥75%, ≥80%, ≥85%, or ≥90%). In some embodiments, to allow breeding, each dog must have at least 2 MEIs in Cfa6.6, Cfa6.7, Cfa6.66, or Cfa6.83. In some embodiments, to allow breeding, the total number of MEIs at those four loci for each dog must be at least 3. In some embodiments, the dogs may have a heterozygosity deficiency at locus Cfa6.6.

In some embodiments, at least one mobile element insertion occurs within at least one gene selected from the group consisting of GTF2I, POM121, and WBSCR17.

In some embodiments, the use of just the genotype at the four loci could be used in a breeding program as a targeted directed goal for which breeding should select dogs from to enhance their desired product (i.e. behavior of a dog), and the method would be free of any other assessment tool. That is, the genotype would be the goal for breeders. If a breeder wanted to augment their assistance dog breeding pool, they would identify dogs with higher copy number of MEIs—either a total copy number, or higher copy numbers at specific loci as discussed above, and selectively breed those in hopes of increasing the “assistance dog aptitude” in their next generations of litters.

Those skilled in the art will recognize or be able to ascertain using no more than routine experimentation, many equivalents to the specific embodiments of the invention described herein. Such equivalents are intended to be encompassed by the following claims

What is claimed:

1. A method for predicting the probability of canine success in a training program, comprising:
(a) genotyping a biological sample from a canine;
(b) determining at least one mobile element insertion copy number within the Williams-Beuren Syndrome (WBS) locus on canine chromosome 6; and
(c) predicting the probability of the canine's success in a training program based on the at least one mobile element insertion copy number.

2. The method according to claim 1, wherein the biological sample is blood, saliva, cerebrospinal fluid, skin, or urine.

3. The method according to claim 1, wherein genotyping the biological sample includes PCR amplification and agarose gel electrophoresis.

4. The method according to claim 1, wherein genotyping the biological sample utilizes at least one primer selected from the group consisting of:
CCCCTTCAGCCAGCATATAA, TTCTCTGGGCTGTCTGGACT, TGGAGCCATGATTAGGAAGG, TAAGGAAGGACCCCATTTCC, TGCTGCTTCATGTTCTGTGA, TGGTGCATTAGCTTTGGTTG, AACCACAGGAACAAAACCTCA, and CCTCCTGTTGGACATTTGGA.

5. The method according to claim 1, wherein the mobile element insertions interrupt a gene in the WBS locus.

6. The method according to claim 5, wherein the mobile element insertions are retrotransposon mobile element insertions.

7. The method according to claim 6, wherein the retrotransposons are short interspersed nuclear elements (SINEs) or a long interspersed nuclear elements (LINEs).

8. The method according to claim 1, wherein at least one mobile element insertion occurs within at least one gene selected from the group consisting of GTF2I, POM121, and WBSCR17.

9. The method according to claim 1, wherein predicting the probability of the canine's success in a training program includes rating the canine on attachment/attention-seeking behaviors and separation-related problems.

10. The method according to claim 9, wherein the rating for attachment/attention-seeking behaviors is based on Cfa6.6 and Cfa6.83, and the rating for separation-related problems is based on Cfa6.6 and Cfa6.7.

11. The method according to claim 10, wherein which loci are used to determine each rating is based on age of the canine.

12. The method according to claim 1, wherein at least one mobile element insertion is found at Cfa6.6, Cfa6.7, Cfa6.66, or Cfa6.83.

13. The method according to claim 1, wherein at least one mobile element insertion is found at Cfa6.6 and Cfa6.7.

14. The method according to claim 1, wherein the probability of the canine's success in a training program is not based on a mobile element insertion copy number of loci Cfa6.66.

15. The method according to claim 1, wherein the probability of the canine's success in a training program is further based on the heterozygosity deficiency at locus Cfa6.6.

16. The method according to claim 1, wherein the probability of the canine's success in a training program is further based on an age of the canine.

17. A method of producing dogs that are more likely to exhibit a sociable behavior comprising:
(a) determining at least one mobile element insertion copy number within the Williams-Beuren Syndrome (WBS) locus on canine chromosome 6 for each of a male and a female dog intended for breeding; and
(b) mating the male and female dog to produce offspring.

18. The method according to claim 17, wherein the dogs have a heterozygosity deficiency at locus Cfa6.6.

19. The method according to claim 17, wherein the at least one mobile element insertion occurs within at least one gene selected from the group consisting of GTF2I, POM121, and WBSCR17.
